(19) United States
(12) Patent Application Publication
(10) Pub. No.: US 2009/0007633 A1
(43) Pub. Date: Jan. 8, 2009

(54) Measuring Device for Measuring the Oxygen Fraction in Respiratory Air

(75) Inventor: Norbert Wittschief, Achim (DE)

Correspondence Address: FITCH, EVEN, TABIN & FLANNERY, P. O. BOX 18415, WASHINGTON, DC, 20036, US

(73) Assignee: ATLAS ELEKTRONIK GmbH., Bremen (DE)

(21) Appl. No.: 12/087,735

(22) PCT Filed: Dec. 12, 2006
(86) PCT No.: PCT/EP2006/011922

§ 371 Date: Jul. 14, 2008

(30) Foreign Application Priority Data
Jan. 28, 2006 (DE) 10 2006 004 051.1

(51) Int. Cl. G01N 33/497

(52) U.S. Cl. 73/23.3

(57) ABSTRACT

A measuring device for measuring the oxygen fraction in respiratory air, especially in aircraft, has an oxygen sensor to which an electrical, analog, sinusoidal input signal with a fixed base frequency is fed and which emits an electrical, analog output signal containing a useful signal component with a double-base frequency and at least one interference signal component with a base frequency, in addition to an evaluation circuit for determining a measured value as a measure of the oxygen fraction, the output signal being fed to the circuit. To improve measuring accuracy, while simultaneously reducing fluctuations in the measured values, the output signal and a sinusoidal reference signal with a base frequency are fed to an adder located between the oxygen sensor and evaluation circuit, the reference signal phase and amplitude being set such that the interference signal component is largely compensated for in the output signal of the adder.

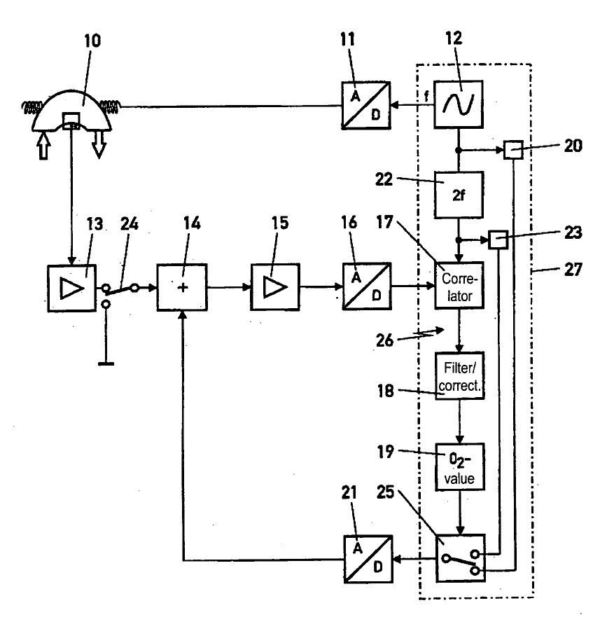

The invention relates to a measuring device for measuring the oxygen fraction in respiratory air, in particular for the supply of respiratory air in aircraft, according to the preamble of claim 1.

In aircraft, for example in fighter jets, oxygen is added to the respiratory air for the aircraft passengers, the amount of oxygen added being dependent on the altitude. The oxygen fraction or oxygen content in the respiratory air is detected using a measuring device and is output as a measured value which, on the one hand, is displayed and, on the other hand, is used as a controlled variable for mixing the respiratory air. A fundamental part of such a measuring device is an oxygen sensor through which the respiratory air flows and which has a magnet coil. A sinusoidal alternating current at a constant fundamental frequency, for example 15 Hz, is supplied to the magnet coil as an input signal of the oxygen sensor. An output signal can be tapped off from the output of the oxygen sensor, which output signal has, on account of a rectifying effect of the oxygen sensor, a useful signal component at twice the fundamental frequency, that is to say 30 Hz in the example, and interference signal components, of which the interference signal component with the greatest amplitude is at the fundamental frequency, that is to say 15 Hz in the example. The output signal from the oxygen sensor is supplied to an evaluation circuit which uses the information which relates to the oxygen fraction and is contained in the useful signal component to determine a measured value which is displayed and/or used to correct the oxygen fraction.

The invention is based on the object of improving the measurement accuracy in the case of a measuring device of the type mentioned initially while simultaneously reducing the fluctuations in the measured values.

According to the invention, the object is achieved by means of the features in claim 1.

The measuring device according to the invention has the advantage that the interference signal component, which is significant in the output signal from the oxygen sensor and is at the fundamental frequency, is suppressed by adding the reference signal to the output signal and the signal-to-noise ratio which is available at the evaluation circuit and is intended to obtain the oxygen measured value is thus considerably improved.

At the same time, the adder which is provided for the purpose of compensating for the interference signal can be used, without additional hardware, to functionally test the measuring device by virtue of the fact that, according to one preferred embodiment of the invention, a test signal at twice the fundamental frequency is connected to the adder instead of the reference signal and the supply of the output signal from the oxygen sensor to the adder is inhibited.

Expedient embodiments of the measuring device according to the invention, with advantageous developments and refinements of the invention, emerge from the further claims.

According to one advantageous embodiment of the invention, both the reference signal and the test signal are generated using a digital sine-wave generator, the reference signal being at the fundamental frequency and the test signal being at twice the fundamental frequency by virtue of frequency doubling. The phase and amplitude of both signals have been set when they are supplied to the adder. The digital signal generator which also provides the analog input signal for the oxygen sensor, which in turn results from digital/analog conversion of the output signal from the digital sine-wave generator, is preferably used to form the reference signal and the test signal.

The invention is described in more detail below using an exemplary embodiment which is illustrated in the drawing. In this case, the drawing shows a block diagram of a circuit structure of the measuring device.

The measuring device which is illustrated in the form of a block diagram and is intended to measure the oxygen fraction in respiratory air has an oxygen sensor 10 through which the respiratory air flows and whose output signal contains an item of information relating to the oxygen fraction of the respiratory air. Such an oxygen sensor is known, for example, by the type name “PATO” from Drager Medical. The oxygen sensor 10 whose known structure is not specifically described in any more detail here has a magnet coil which is supplied with a sinusoidal alternating current at a constant frequency. This alternating current represents an electrical, analog, sinusoidal input signal for the oxygen sensor 10, said signal being tapped off from a digital/analog converter 11 whose input is connected to the output of a digital sine-wave generator 12. The sine-wave generator 12 generates a digital sinusoidal signal at the fundamental frequency 1f which is 15 Hz, for example. An electrical, analog output signal which, on account of a rectifying effect of the oxygen sensor 10, contains a useful signal component at twice the fundamental frequency 2f and interference signal components is present at the output of the oxygen sensor 10. Of the interference signal components, one interference signal component at the fundamental frequency 1f is particularly significant, that is to say it has a large amplitude which allows only an extremely poor signal-to-noise ratio for evaluating the output signal.

In order to largely suppress this interference signal component at the fundamental frequency 1f, the output signal from the oxygen sensor 10, which is amplified in a preamplifier, is supplied to an adder 14. The adder 14 is also supplied with an analog, digital, sinusoidal reference signal at the fundamental frequency 1f, the amplitude and phase of which have been set in such a manner that the interference signal component is largely compensated for at the output of the adder 14. For this purpose, the reference signal must essentially be applied to the adder 14 in phase opposition to the interference signal and with approximately the same amplitude. The output signal from the adder 14 is supplied to an evaluation circuit 26 via an amplifier 15 and an analog/digital converter 16. The evaluation circuit 26 has a correlator 17 which is allocated, on the one hand, the output signal from the analog/digital converter 16 and, on the other hand, the digital sinusoidal signal whose frequency has been doubled using a frequency doubler 22. The digital output signal from the correlator 17 passes through a filter and correction element 18 in which the pressure and temperature dependence of the oxygen sensor 10 is compensated for. The amplitude of the output signal from the filter and correction element 18 is a measure of the oxygen content of the respiratory air flowing through the oxygen sensor 10. The oxygen measured value is determined and displayed in block 19 and/or is used as a control signal for correcting and/or setting the oxygen fraction in the respiratory air.

The analog, sinusoidal reference signal at the fundamental frequency 1f, which is supplied to the adder 14, is generated in such a manner that the amplitude and phase of the digital sinusoidal signal from the sine-wave generator 12 are set in the module 20 and said digital sinusoidal signal is applied to a digital/analog converter 21. The analog reference signal at the fundamental frequency 1f can be tapped off from the output of the digital/analog converter 21. The amplitude and phase of the digital sinusoidal signal are correctly set once in an adjustment or calibration operation and are invariably fixed in the measuring device.

A self-test function can be integrated in the measuring device without additional outlay in terms of hardware. For this purpose, an analog, sinusoidal test signal at twice the fundamental frequency 2f is applied to the adder 14 instead of the amplified, analog output signal from the oxygen sensor 10. This test signal whose amplitude corresponds to a desired oxygen fraction can be used to test the entire circuit, which is arranged downstream of the oxygen sensor 10, for correct functionality. The disconnection of the output signal of the oxygen sensor 10 from the adder 14 is symbolically illustrated using the switch 24 which connects one input of the adder 14 to zero potential during changeover. The test signal is derived from the digital sinusoidal signal from the sine-wave generator 12 at twice the fundamental frequency 2f, which sinusoidal signal is tapped off from the output of the frequency doubler 22 and the phase and amplitude of which are set in the module 23. This digital sinusoidal signal is supplied to the digital/analog converter 21. In order to symbolize the operation of changing over from the digital sinusoidal signal which is used to obtain the reference signal and is at the fundamental frequency 1f to the digital sinusoidal signal which is used to obtain the test signal and is at twice the fundamental frequency 2f, a changeover switch 25 which is arranged upstream of the digital/analog converter 21 and has one switch output and two switch inputs is illustrated in the block diagram of the measuring device. The switch output is connected to the input of the digital/analog converter 21, while the digital sinusoidal signal whose amplitude and phase have been set and which is at the fundamental frequency 1f is present at one switch input, and the digital sinusoidal signal whose amplitude and phase have been set and which is at twice the fundamental frequency 2f is present at the other switch input.

The functions of the evaluation circuit 26 having the modules of correlator 17, filter and correction element 18 and block 19, of the digital sine-wave generator 12, of the frequency doubler 22, of the modules 20 and 23 for amplitude and phase setting, and of the changeover switch 25 are implemented using a processor 27 or a plurality of processors.

1. A measuring device for measuring the oxygen fraction in respiratory air, in particular for the supply of respiratory air in aircraft, said device having an oxygen sensor (10) which is supplied with an electrical, analog, sinusoidal input signal at a fixed fundamental frequency (1f) and outputs an electrical, analog output signal which has a useful signal component at twice the fundamental frequency (2f) and at least one interference signal component at the fundamental frequency (1f), and having an evaluation circuit (26) for determining a measured value as a measure of the oxygen fraction, which evaluation circuit is supplied with the output signal from the oxygen sensor (10), characterized in that an adder (14) is connected between the oxygen sensor (10) and the evaluation circuit (26) and is supplied with, on the one hand, the output signal and, on the other hand, a sinusoidal reference signal at the fundamental frequency (1f), and in that the phase and amplitude of the reference signal are set in such a manner that the at least one interference signal component at the fundamental frequency (1f) is largely compensated for in the output signal from the adder (14).

2. The measuring device as claimed in claim 1, characterized in that the analog output signal from the oxygen sensor (10), which is supplied to the adder (14), passes through a preamplifier (13), and in that an amplifier (15) is arranged at the output of the adder (14), and a digital/analog converter (16) is arranged between the amplifier (15) and the evaluation circuit (26).

3. The measuring device as claimed in claim 1, characterized in that the analog input signal for the oxygen sensor (10) is tapped off from a digital/analog converter (11) which is supplied with a digital sinusoidal signal at the fundamental frequency (1f), which is tapped off from the output of a digital sine-wave generator (12).

4. The measuring device as claimed in claim 3, characterized in that the analog reference signal is tapped off from the output of a digital/analog converter (21) which is supplied with the digital sinusoidal signal at the fundamental frequency (1f) which is tapped off from the digital sine-wave generator (12) and whose amplitude and phase have been set.

5. The measuring device as claimed in claim 2, characterized in that the evaluation circuit (26) has a correlator (17) which is supplied with, on the one hand, the output signal from the digital/analog converter (16) and, on the other hand, the digital sinusoidal signal at twice the fundamental frequency (2f) and the output of which has a filter and correction element (18) connected downstream of it.

6. The measuring device as claimed in claim 1, characterized in that the adder (14) can be alternately allocated, on the one hand, the output signal from the oxygen sensor (10) and the reference signal and, on the other hand, only a sinusoidal test signal at twice the fundamental frequency (2f).

7. The measuring device as claimed in claim 6, characterized in that an electrical changeover switch (25) having one switch output, which is connected to the digital/analog converter (21), and two switch inputs, which can be alternately changed over to the switch output, is arranged upstream of the digital/analog converter (21) that is connected to the adder (14), in that the digital sinusoidal signal whose amplitude and phase have been set and which is at the fundamental frequency (1f) is present at the first switch input, and the digital sinusoidal signal whose amplitude and phase have been set and which is at twice the fundamental frequency (2f) is present at the second switch input, and in that the oxygen sensor (10) is disconnected from the adder (14) when the switch output is connected to the second switch input.

8. The measuring device as claimed in claim 1, characterized in that the functions of the evaluation circuit (26), of the digital sine-wave generator (12), of the frequency doubling of the digital sinusoidal signal, of the phase and amplitude setting of the digital sinusoidal signal at the fundamental frequency (1f) and the digital sinusoidal signal at twice the fundamental frequency (2f), and of the electronic changeover switch (25) are implemented using at least one processor (27).
